(19) United States
(12) Patent Application Publication
(10) Pub. No.: US 2008/0064705 A1
(43) Pub. Date: Mar. 13, 2008

(54) Theophylline-based nitophenylpiperazine derivatives for enhancing aortic smooth muscle relaxation

(75) Inventor: Ing-Jun Chen, Kaohsiung City (TW)

Correspondence Address: TROJAN LAW OFFICES, 9250 WILSHIRE BLVD, SUITE 325, BEVERLY HILLS, CA, 90212, US

(73) Assignee: KAOHSIUNG MEDICAL UNIVERSITY

(21) Appl. No.: 11/520,174

(22) Filed: Sep. 12, 2006

(51) Int. Cl. A61K 31/522; C07D 473/02

(52) U.S. Cl. 514/252.16; 544/270

(57) ABSTRACT

A substance for enhancing an aortic smooth muscle relaxation is provided. The substance is one selected from the group consisting of a compound of formula (I), a pharmaceutical acceptable therefrom and a solvate therefrom,

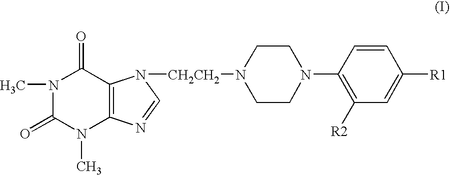

wherein either of R1 and R2 is one of a hydrogen and a nitro group.

### FIELD OF THE INVENTION

The present invention relates to a theophylline-based nitrophenylpiperazine derivative being a vasorelaxant, and more particularly to a theophylline-based nitrophenylpiperazine derivative for enhancing the relaxation of aortic smooth muscle.

### BACKGROUND OF THE INVENTION

In recent years, a series of xanthine-based vasorelaxants have been synthesized, wherein KMUP-1 and KMUP-2 have been demonstrated to possess novel pharmacological activities by raising the contents of cyclic nucleotides, inhibiting phosphodiesterases (PDEs), and activating K⁺ channels, thus, resulting in relaxations in aortic, corpora cavernosa, and tracheal smooth muscles. Currently, it is found that KMUP-1 activates large-conductance Ca²⁺-activated K⁺ (BK_{Ca}) channel activity in rat basilar arteries through protein kinase-dependent and -independent mechanisms.

Soluble guanylyl cyclase (sGC) is an important target for endogenous nitric oxide (NO), NO-releasing drugs such as glyceryl trinitrate, and novel substances such as YC-1 or BAY 41-2272 that sensitize the enzyme for activation by NO. The novel sGC activator, YC-1, has attracted much attention recently because of its unique action profile, making it and related drugs promising therapeutic tools for the treatment of endothelial dysfunction without the accompanying of the adverse effects of classic nitrovasodilators. The positive effects of YC-1 seem to result from multiple actions of the drug, including NO-independent activation of sGC, promotion of cGMP formation, antiproliferation of VSM cells, and inhibition of PDE-catalyzed cGMP hydrolysis. Recently, it has been reported that KMUP-1 and KMUP-2, like YC-1, possess multiple pharmacological activities on smooth muscles. In addition, various pharmacologic agents such as statins, estrogen, and insulin were described to have endothelium-dependent or endothedium nitro oxide synthase (eNOS) activation activities, leading to modulation of vascular function. Thus, new discoveries of eNOS activators are insightful in clinics for the treatment of patients with vascular dysfunction.

So far, vascular relaxation is focused on the role of the endothelium in reducing tone of VSM by release of NO, activation of sGC, and subsequent increases in cGMP. An alternative second messenger pathway that plays a crucial role in eliciting relaxation of vascular smooth muscle (VSM) may involve receptor-mediated activation of adenylyl cyclase (AC) and formation of cAMP. Usually the NO/cGMP pathway is considered to be endothelium-dependent, whereas cAMP-elevating pathways are not. Intracellular cyclic nucleotides are involved in the control of smooth muscle tone, and degraded by PDEs. As a consequence, PDE activity is also implicated in the modulation of smooth muscle tone, and thus, inhibition of PDE can lead to smooth muscle relaxation.

On the other hand, K⁺ channels play a major role in the regulation of the resting membrane potential and modulate vascular smooth muscle tone. Endothelium-derived hyperpolarizing factor (EDHF) activates the K⁺ channels, and the subsequent K⁺ flux hyperpolarizes the cell membrane to relax the smooth muscle cell. It is suggested that activation of endothelium K_{ATP} channels may also release endothelium-derived NO or EDHF. NO donors have been shown to activate K_{ATP} channels via a cGMP-dependent mechanism in rat aortic smooth muscle cells and rabbit mesenteric artery, and by a cGMP-independent mechanism in rat mesenteric artery.

However, an electron-withdrawing group such as a nitro or chloro group on a benzene ring is suggested to have important pharmacological activities compared with the electron-donating group that has been demonstrated in KMUP-2. The mentioned interesting results further encourage us to design many KMUP-1 analogues, and therefore both KMUP-3 (7-[2-[4-(4-nitrobenzene)-piperazinyl]ethyl]-1,3-dimethylxanthine) and KMUP-4 (7-[2-[4-(2-nitrobenzene)piperazinyl]ethyl]-1,3-dimethylxanthine) were developed, where the chemical structures thereof have para- and ortho-nitro groups respectively which are strikingly different from the ortho-chloro-substituted KMUP-1 (please refer to FIG. 1). The U.S. Pat. No. 6,979,687 has disclosed the effects of KMUP-1 and KMUP-2 on CCSM (corpus cavernosum smooth muscle), which retain the cyclic nucleotide elevation, promote K⁺-channel activation, and enhance PDEs inhibition activities.

Based on the above, to develop more potent eNOS activators than YC-1, also accompanying with cGMP-enhancing activity, KMUP-3 and KMUP-4 are synthesized to further investigate how the structure-activity relationship thereof affects the relaxation on vascular smooth muscles.

From the above description, it is known that how to develop theophylline-based derivatives for enhancing aortic smooth muscle relaxation has become a major problem to be solved. In order to overcome the drawbacks in the prior art, novel theophylline-based derivatives for enhancing aortic smooth muscle relaxation are provided. The particular design in the present invention not only solves the problems described above, but also is easy to be implemented. Thus, the invention has the utility for the industry.

### SUMMARY OF THE INVENTION

The present invention provides the possible mechanisms of vascular smooth muscle relaxations induced by KMUP-3 and KMUP-4, the abilities thereof to enhance cyclic nucleotides, endothelium-dependent and K⁺-channel activities, and PDEs inhibiting activities. Furthermore, the present invention provides vasorelaxant agents, such as KMUP-3 and KMUP-4, which are of use in the future management of VSM dysfunction.

In accordance with one aspect of the present invention, a substance for enhancing an aortic smooth muscle relaxation is provided. The substance is one selected from the group consisting of a compound of formula (I), a pharmaceutical acceptable therefrom and a solvate therefrom,

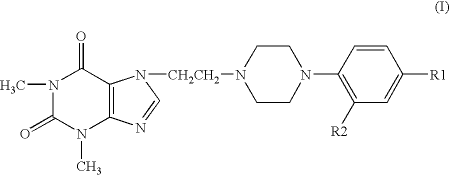

wherein either of R1 and R2 is one of a hydrogen and a nitro group.

Preferably, the R1 group is a nitro group.

Preferably, the R2 group is a nitro group.

In accordance with another aspect of the present invention, a pharmaceutical composition for enhancing an aortic smooth muscle relaxation is provided. The pharmaceutical composition includes a substance of claim 1 and one selected from the group consisting of a pharmaceutical excipient, a diluent and a carrier.

In accordance with a further aspect of the present invention, a method for synthesizing a compound for enhancing an aortic smooth muscle relaxation is provided. The method comprises the following steps: (1) dissolving theophylline into 1,2-dibromoethane to form a mixture, (2) adding NaOH into the mixture to obtain an oil-like solution, (3) purifying the oil-like solution to obtain an oil-like compound, (4) adding piperazine to react with the oil-like compound to obtain a first coarse crystal, (6) recrystallizing and purifying the first coarse crystal to obtain a first crystal compound, (6) dissolving the first crystal compound in a solvent to form a first solution, (7) dissolving 2-chloronitrobenzene and 4-chloronitrobenzene respectively in the first solution to form a second solution, and (8) obtaining the compound from the second solution.

Preferably, the reaction temperature of NaOH reacting with the first solution is performed at approximately 90-120° C.

Preferably, the reaction temperature of NaOH reacting with the first solution is performed at 100° C.

Preferably, the recrystallization buffer is methanol.

Preferably, the purification is performed by a column.

Preferably, the column has a packing gel being silica gel 60.

Preferably, the solvent of the second crystal compound is methanol.

Preferably, the compound is obtained by performing a refluxation.

In accordance with further another aspect of the present invention, a method for enhancing an aortic smooth muscle relaxation of a mammal is provided. The method comprises a step of administrating to the mammal one of the group consisting of a compound of formula (I), a salt thereof and a solvate thereof, and a pharmaceutical carrier,

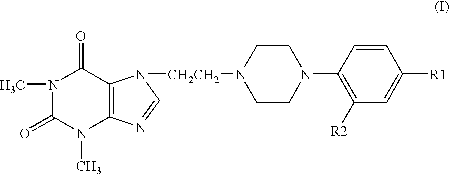

wherein either of R1 and R2 is one of a hydrogen and a nitro group.

The above aspects and advantages of the present invention will become more readily apparent to those ordinarily skilled in the art after reviewing the following detailed descriptions and accompanying drawings, in which:

### BRIEF DESCRIPTION OF THE DRAWINGS

FIG. 1 shows the respective chemical structures of KMUP-3 and KMUP-4 according to the present invention;

FIG. 2 shows the respective aortic relaxation diagrams induced by cumulative concentration of KMUP-3 and KMUP-4 in endothelium-intact (E⁺) and endothelium-denuded (E⁻) rat aortic rings precontracted with phenylephrine (PE) according to the present invention;

FIG. 3 is an aortic relaxation diagram induced by cumulative concentration of KMUP-3, KMUP-4, theophylline, milrinone, rolipram, zaprinast (1 nM-100 μM) on PE-precontracted endothelium-intact aortic rings respectively according to the present invention;

FIG. 4 shows the respective aortic relaxation diagrams induced by cumulative concentration of KMUP-3 and KMUP-4 (1 nM-100 μM) on endothelium-intact aortic rings precontracted with 10 μM PE or 80 mM K⁺ solution according to the present invention;

FIG. 5 shows the respective aortic relaxation diagrams induced by cumulative concentration of KMUP-3 and KMUP-4 (1 nM-100 μM) on endothelium-intact aortic rings precontracted with PE (10 μM) in the absence or presence of various of K⁺ channel blockers according to the present invention;

FIGS. 6(A)-6(D) show the respective aortic relaxation diagrams induced by cumulative concentration of KMUP-3 and KMUP-4 (1 nM-100 μM) on endothelium-intact and endothelium-denuded aortic rings precontracted by PE (10 μM) in the absence or presence of L-NAME (100 μM), ODQ (1 μM), SQ 22536 (100 μM), indomethacin (10 μM) or ODQ+SQ 22536 according to the present invention;

FIG. 7 shows the respective aortic relaxation diagrams induced by cumulative concentration of KMUP-3 and KMUP-4 (1 μM) on SNP- and isoproterenol-induced vasorelaxants in endothelium-denuded aortic rings precontracted by PE (10 μM) according to the present invention; and

FIG. 8 is a densitometry result of the expression of eNOS in HUVECs induced by YC-1, KMUP-3, and KMUP-4 (0.1 μM) according to the present invention.

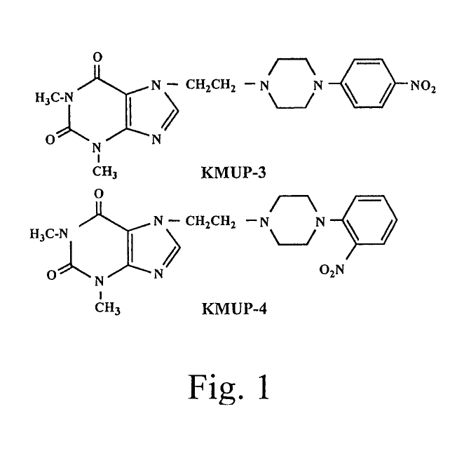

### DETAILED DESCRIPTION OF THE PREFERRED EMBODIMENT

The present invention will now be described more specifically with reference to the following embodiments. It is to be noted that the following descriptions of preferred embodiments of this invention are presented herein for the purposes of illustration and description only; it is not intended to be exhaustive or to be limited to the precise form disclosed.

The present invention discloses a serial of the theophylline-based nitrophenylpiperazine derivatives chemically with formula I for enhancing an aortic smooth muscle relaxation.

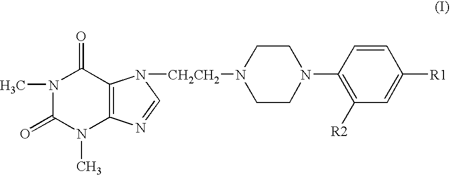

The endothelium plays a role in determining vascular tone by the release of vasoactive factors such as NO, prostacyclin, and EDHF. In general, the endothelium releases predominantly NO in large arteries such as rabbit carotid artery, although the contribution of EDHF assumes importance in smaller resistance arteries such as rat mesenteric artery. It is widely accepted that NO plays a central role in regulating the function of vascular smooth muscles. Its direct effect on vascular tone is brought about by cGMP formation in vascular smooth muscle cells. Several NO-related compounds, such as SNP and endogenous NO, activate sGC, elevate cGMP, and relax smooth muscles. The main finding of the present invention is that the vasorelaxant mechanisms of two KMUP-1 analogues, KMUP-3 and KMUP-4, are through NO/sGC/cGMP, AC/cAMP, and endothelium-dependent pathways, K⁺ channels, and PDE inhibitory activities.

KMUP-3 and KMUP-4 produce concentration-dependent aortic relaxations against the contraction induced by PE in endothelium-intact aortic rings. In endothelium-denuded aortic rings or during the inhibition of NOS, KMUP-3 and KMUP-4 are still able to evoke vasorelaxant effects. Therefore, it is suggested that KMUP-3 and KMUP-4 are more likely to have a direct action on a vascular smooth muscle component that does not involve the endothelium. However, at least part of the response to KMUP-3 or KMUP-4 is endothelium-dependent because a significant downward shift in the concentration-response curve is observed after endothelium removal. This concept is further supported by the evidence that KMUP-3 and KMUP-4 enhance the expression of eNOS in HUVECs. Increased eNOS protein is also found in a representative sGC activator YC-1, which appears to be less potent than KMUP-3 or KMUP-4. Considering the pEC₅₀ and Eₘₐₓ values obtained from endothelium-denuded aortic rings, it is suggested that the vascular smooth muscle relaxant response, not interacting with endothelium, is more potent in KMUP-4 than in KMUP-3.

In endothelium-intact aortic rings, the vasorelaxant responses of KMUP-3 and KMUP-4 are attenuated by pretreatment with ODQ, the sGC inhibitor, or SQ 22536, the AC inhibitor, and they are also reduced by ODQ or SQ 22536 in endothelium-denuded aorta. Additionally, the relaxations of both agents are dramatically reduced by treating ODQ with SQ 22536 in endothelium-intact and endothelium-denuded aorta, but they are still not completely inhibited. The results of the present invention indicate that the relaxations of KMUP-3 and KMUP-4 not only activate the sGC/cGMP, AC/cAMP, and endothelium-dependent pathways, but they also have another direct action on vascular smooth muscles, which could involve K⁺-channel activation. Because the reduction of KMUP-3 relaxation by combined ODQ and SQ 22536 is more obvious than that of KMUP-4, it is suggested that KMUP-3, in contrast to KMUP-4, mainly acts on the cyclic nucleotide pathway, whereas KMUP-4 acts mainly on the vascular smooth muscle contractile mechanisms and/or the K⁺ channels.

Previous studies have shown that the vasodilators dependent on the K⁺-channel mechanism reduce their relaxant effects when exposed to high-K⁺ solutions because an increase in extracellular K⁺ attenuates the K⁺ gradient across the plasma membrane, thus rendering the K⁺ channel-activating mechanism ineffective; indeed, similar results were also found for KMUP-1 and KMUP-2. In the present invention, it is demonstrated that the vasorelaxations elicited by KMUP-3 and KMUP-4 are significantly reduced by increasing the extracellular concentration of K⁺ (80 mM). Accordingly, it is suggested that the relaxations of KMUP-3 and KMUP-4 involve smooth muscle hyperpolarization, and this action is more marked in KMUP-4 than in KMUP-3. The results further support the vasorelaxant action of KMUP-4 is predominantly on the K⁺-channel activation. Again, it is examined the contribution of K⁺-channels to KMUP-3- and KMUP-4-induced vasorelaxations in the following.

The vasorelaxant effects of KMUP-3 and KMUP-4 are significantly decreased by the above K⁺ channel blockers. The following results confirm that K⁺-channel activation also plays an important role on KMUP-3- and KMUP-4-induced vasorelaxations, especially for KMUP-4. KMUP-3 and KMUP-4 enhances the vasorelaxant responses not only to a cGMP-dependent vasodilator SNP but also to a cAMP-dependent vasodilator isoproterenol. On the other hand, it is observed that KMUP-3 and KMUP-4 also affect cyclic nucleotide breakdown at 10 mM because they inhibit the enzyme activities of PDE3, PDE4, and PDE5.

Furthermore, KMUP-3 and KMUP-4 significantly raise the intracellular cGMP and cAMP levels in RASMCs. Increased cGMP and cAMP levels are also markedly reduced by ODQ and SQ 22536, respectively. Therefore, it is suggested that KMUP-3 and KMUP-4 activate both NO/sGC/cGMP and AC/cAMP pathways and inhibit PDEs and thereby augment the intracellular cGMP and cAMP contents, leading to aortic smooth muscle relaxations.

In summary, the results of the present invention provide the evidence that the vascular smooth muscle relaxant activities of KMUP-3 and KMUP-4, KMUP-1 analogues, are most likely via cyclic nucleotide elevation, indomethacin-sensitive endothelium activation, K⁺-channel stimulation, and PDE inhibition. Although KMUP-3 and KMUP-4 show the effects similar to those of KMUP-1, neither AC/cAMP nor PG pathways were involved by KMUP-1 in rat vascular smooth muscles. Here, it is suggested that KMUP-3 and KMUP-4 raise cyclic nucleotides partly through PDE inhibition, produce vasorelaxing prostanoids, and therefore stimulate the K⁺ efflux, resulting in attenuation of Ca²⁺ influx-associated contractility in vascular smooth muscles. Because KMUP-3 and KMUP-4 have nonselective PDE-inhibitory activities, indeed, both of KMUP-3 and KMUP-4 act not only on vascular smooth muscle but also on non-vascular smooth muscle.

Pharmaceutical Activity

The pharmaceutical activities of the compounds, KMUP-3 and KMUP-4, of this invention have been proven by the following pharmaceutical experiments.

Wistar rats were provided from the National Laboratory Animal Breeding and Research Center (Taipei, Taiwan). They were housed under conditions of constant temperature and controlled illumination (light on between 7:30 and 19:30). Food and water were available ad libitum. The study was approved by the Animal Care and Use Committee of the Kaohsiung Medical University.

Sildenafil citrate was kindly supplied by Cadila Healthcare Ltd. (Maninagar, India). Apamin, 4-aminopyridine (4-AP), charybdotoxin (ChTX), glibenclamide, indomethacin, Nv-nitro-L-arginine methyl ester (L-NAME), 1H-[1,2,4]oxadiazolo[4,3-a]quinoxalin-1-one (ODQ), 9-(terahydro-2-furanyl)-9H-purin-6-amine (SQ 22536), tetraethylammonium (TEA), and YC-1 were all obtained from Sigma-Aldrich Chemical Co (St Louis, Mo.). All other reagents used were from E. Merck (Darmstadt, Germany). All drugs and reagents were dissolved in distilled water unless otherwise noted. Apamin was dissolved in 0.05 M acetic acid; indomethacin was dissolved in 100 mM sodium carbonate; ChTX, glibenclamide, ODQ, and YC-1 were dissolved in DMSO at 10 mM; KMUP-3 and KMUP-4 were dissolved in 10% absolute alcohol, 10% propylene glycol, and 2% 1 N HCl at 10 mM. Serial dilutions were made in distilled water.

Rat aortic smooth muscles were obtained as sterile surgical specimens. The tissue was washed and cut into 1- to 2-mm pieces and placed into culture dishes with Dulbecco modified Eagle medium (DMEM) containing 20% fetal bovine serum (FBS), 100 U/mL penicillin G, 100 mg/mL streptomycin, and 2 mM glutamine. After these explants attached to the culture dish, usually in 1 to 2 days, DMEM supplemented with 10% FBS, penicillin, streptomycin, and glutamine was added. Rat aortic smooth muscle cells (RASMCs) migrated from the explants in 3-5 days. At this time, the explants were removed, and cells were allowed to achieve confluence. Cells were detached using 0.05% trypsin and 0.02% EDTA at 37° C. for 5 minutes to establish secondary cultures. Cultures were maintained for no more than 4 passages. To exclude contamination by endothelial cells and fibroblasts, the cell homogeneity was further confirmed by the presence of smooth muscle-specific α-actin and α-myosin. Indirect immunofluorescence staining for a variety of antigens was carried out by first plating the cells on chamber slides, fixing the cells in 3.7% formaldehyde in phosphate-buffered saline for 10 minutes, and then permeabilizing the cells with phosphate-buffered saline plus 0.1% Triton X-100. Cells were stained with a mouse monoclonal antibody directed against the amino-terminal 10 amino acids of a-smooth muscle actin and α-myosin (Boehringer Mannheim, Indianapolis, Ind.). All were stained with fluoresceinlabeled goat anti-mouse IgG antibody. Over 95% of the cell preparation was found to be composed of smooth muscle cells.

The aortic rings were prepared as follows. 24 Rats (200-300 g) were killed under mild anesthesia with ether, and their aortas were quickly excised. Thoracic aortas were cleaned of fat and connective tissue and cut into 3- to 4-mm-wide transverse rings, which were then mounted in a 10-mL organ bath and bathed at 37° C. in Krebs solution (NaCl: 118 mM, KCl: 4.8 mM, CaCl₂: 2.5 mM, MgSO₄: 1.2 mM, KH₂PO₄: 1.2 mM, NaHCO₃: 24 mM, glucose: 11 mM), bubbled with a 95% O₂+5% CO₂ mixture.

Isometric tension was recorded with a force displacement transducer (Grass, Model FT03, Quincy, Mass.). The endothelium layer was removed mechanically by inserting the tip of a pair of forceps into the lumen and rolling the tissue back and forth several times on a paper towel moistened with physiological salt solution. At the beginning of each experiment, aortic rings were stretched to a resting tension of 1.5 g and then contracted with phenylephrine (PE, 10 mM), and once the contractions had reached a plateau, the endothelial integrity of the preparations (abbreviated as endothelium-intact in the following) or absence of endothelium (abbreviated as E⁻ in the following) was verified by adding ACh (1 mM) to the superfusate. Only the aortic rings with a vasorelaxant response of >70% inhibition of preconstruction were considered endothelium-intact. The preparations were then washed and allowed to equilibrate with Krebs solution for 45 minutes before being contracted a second time with PE. When the stable vasoconstriction to PE (10 μM) was reached, concentration-response curves to KMUP-3 and KMUP-4 (1 nM-100 μM) were constructed. Additionally, aortic rings were preconstricted with 80 mM KCl. When the contraction reached a steady state, cumulative concentration-response curves to KMUP-3 and KMUP-4 (1 nM-100 μM) were determined. In this experiment, the high-K⁺ solution was prepared by replacing NaCl with KCl (80 mM) in an equimolar amount. In another experiment, the effects of KMUP-3 and KMUP-4 on the vasorelaxant responses to isoproterenol, a β-adrenoceptor-mediated cAMP-dependent vasodilator, or SNP, a NO-donor/cGMP-dependent vasodilator, were investigated by incubating the E⁻ aortic rings with 1 mM of KMUP-3 and KMUP-4 respectively for 30 minutes before either isoproterenol (0.01 μM-10 mM) or SNP (1 nM-1 μM) was added.

To examine the possible mechanisms of vasorelaxant effects of KMUP-3 and KMUP-4, the aortic rings were pretreated with the sGC inhibitor ODQ (1 μM), the NOS inhibitor L-NAME (100 μM), the AC inhibitor SQ 22536 (100 μM), the PG inhibitor indomethacin (10 μM), the nonselective K⁺ channel blocker TEA (10 mM), the K_{ATP} channel blocker glibenclamide (1 μM), the voltage-dependent K⁺ (K_{V}) channel blocker 4-AP (100 μM), the small-conductance Ca²⁺-dependent K⁺ (SK_{Ca}) channel blocker apamin (1 μM), or the BK_{Ca} channel blocker ChTX (0.1 μM) for 30 minutes before the addition of KMUP-3 or KMUP-4.

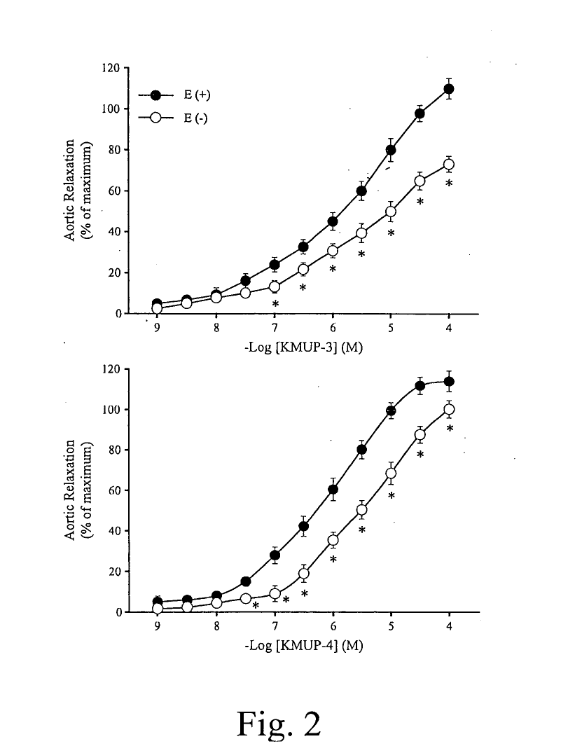

Please refer to FIG. 2. FIG. 2 shows the respective aortic relaxation diagrams induced by cumulative concentration of KMUP-3 and KMUP-4 in endothelium-intact and endothelium-denuded rat aortic rings precontracted with PE (10 μM) according to the present invention. The concentration of KMUP-3 and KMUP-4 is from 1 nM to 100 μM. Both of KMUP-3 and KMUP-4 induced relaxant response are significantly different in endothelium-intact and endothelium-denuded (E⁻) aortic rings. Each point represents the mean±SEM of 8-10 experiments. According to the illustration in FIG. 2, cumulative concentrations of KMUP-3 and KMUP-4 (1 nM-100 μM) produce concentration-dependent vasorelaxations in endothelium-denuded aorta (E⁻) with pEC₅₀ values of 5.08±0.03 and 5.94±0.09, respectively, and in endothelium-intact aorta with pEC₅₀ values of 6.21±0.10 and 6.45±0.12, respectively, indicating that the relaxant responses of both KMUP-3 and KMUP-4 are likely to be endothelium-independent. Also, under the E⁻ condition, the maximal relaxation (Eₘₐₓ) values of KMUP-3 and KMUP-4 are 73±3.9% and 99±4.3%, respectively, suggesting that KMUP-4 seems to be more endothelium-independent than KMUP-3. However, in the presence of either KMUP-3 or KMUP-4, there was a significant shift in the response curve after endothelium removal, suggesting that at least part of the observed effect is endothelium-dependent.

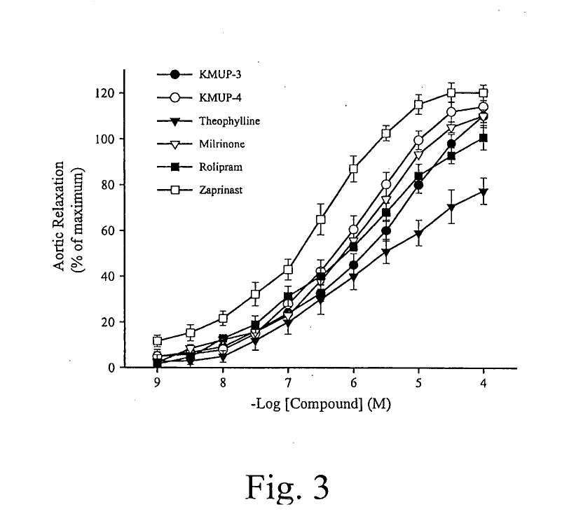

Furthermore, in order to compare the relaxant effects of KMUP-3 and KMUP-4 to the existing commercial agents, we choose theophylline which is the nonselective PDE inhibitor, milrinone which is the PDE3 selective inhibitor, rolipram which is the PDE4 selective inhibitor, and zaprinast which is the PDE5 selective inhibitor, as contrast agents. Please refer to FIG. 3, which shows the relaxant effects of KMUP-3, KMUP-4 and the mentioned agents and each point represents the mean±SEM of 8-10 experiments. The concentration of KMUP-3, KMUP-4, and theophylline, milrinone, rolipram, and zaprinast is from 1 nM to 100 μM. Based on the illustration in FIG. 3, it is calculated that the pEC₅₀ values of theophylline are 5.45±0.13, the pEC₅₀ values of milrinone are 6.34±0.07, the pEC₅₀ values of rolipram are 6.39±0.05, and the pEC₅₀ values of zaprinast are 6.95±0.11. According to the above, the pEC₅₀ values of KMUP-3 and KMUP-4 are 6.21±0.10 and 6.45±0.12 respectively, suggesting that KMUP-3 and KMUP-4 have the similar vasorelaxant effect.

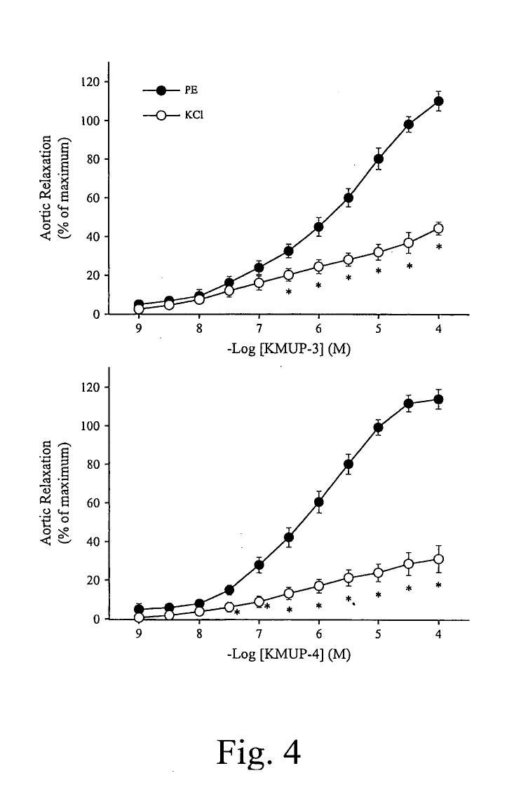

Please refer to FIG. 4. FIG. 4 shows the respective aortic relaxation diagrams induced by cumulative concentration of KMUP-3 and KMUP-4 (1 nM-100 μM) on endothelium-intact aortic rings precontracted with 10 μM PE or 80 mM K⁺ solution according to the present invention. KMUP-3 and KMUP-4 (1 nM-100 μM) caused concentration-dependent relaxations in PE (10 μM)-contracted endothelium-intact rat thoracic aorta. By contrast, both of KMUP-3 and KMUP-4 had a great reduction of relaxation in the presence of 80 mM K⁺ solution, more prominent in KMUP-4.

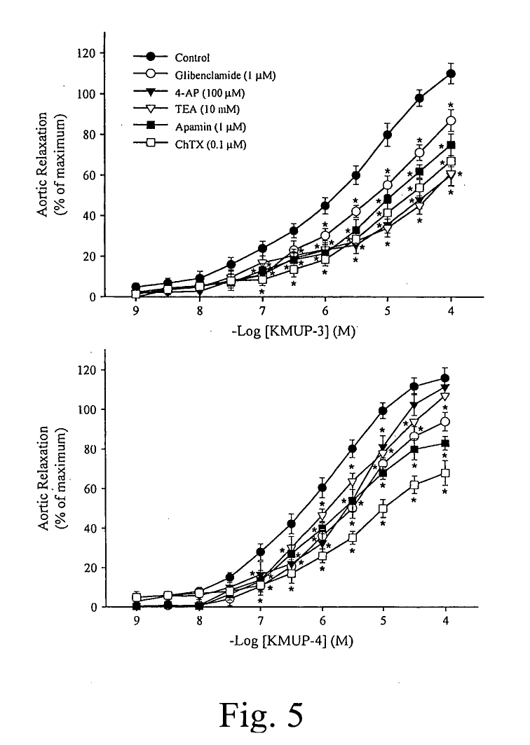

Please refer to FIG. 5. FIG. 5 shows the respective aortic relaxation diagrams induced by cumulative concentration of KMUP-3 and KMUP-4 (1 nM-100 μM) on endothelium-intact aortic rings precontracted with PE (10 μM) in the absence or presence of various of K⁺ channel blockers according to the present invention. Aortic relaxations of KMUP-3 and KMUP-4 were reduced by TEA (10 mM), which is the nonselective K⁺ channel blocker, glibenclamide (1 μM), the K_{ATP} channel blocker, 4-AP (100 μM), the K_{V} channel blocker, apamin (1 μM), which is the SK_{Ca} channel blocker, and ChTX (0.1 μM), the BK_{Ca} channel blocker. Nevertheless, the vasorelaxations of KMUP-4 at concentrations up to 30 and 100 μM were not significantly affected by 4-AP and TEA, respectively.

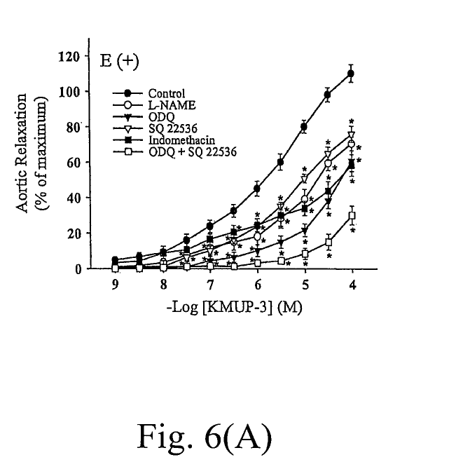

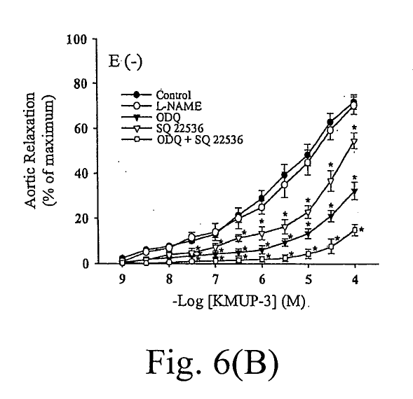

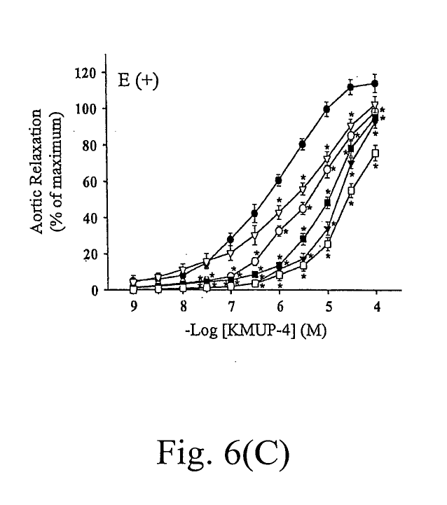

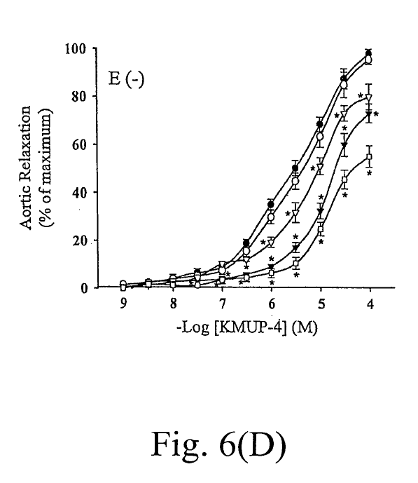

Please refer to FIGS. 6(A)-6(D). FIGS. 6(A)-6(D) show the respective aortic relaxation diagrams induced by cumulative concentration of KMUP-3 and KMUP-4 (1 nM-100 μM) on endothelium-intact and E⁻ aortic rings precontracted by PE (10 μM) in the absence or presence of L-NAME (100 μM), ODQ (1 μM), SQ 22536 (100 μM), indomethacin (10 μM) or ODQ+SQ 22536 according to the present invention. According to the illustrations in FIGS. 6(A) and 6(C), the vasorelaxant responses of KMUP-3 and KMUP-4 are reduced by pretreatment with the NOS inhibitor L-NAME, the sGC inhibitor ODQ, the AC inhibitor SQ 22536, or the PG inhibitor indomethacin in endothelium-intact rat thoracic aorta. Correspondingly, the relaxations of KMUP-3 and KMUP-4 are also attenuated by ODQ or SQ 22536 in E⁻ rat thoracic aorta, but KMUP-3 and KMUP-4 do not exhibit any significant inhibition by L-NAME based on the illustrations in FIGS. 6(B) and 6(D).

To investigate whether the inhibition of the NO/cGMP pathway results in cross-reduction of the AC/cAMP dependent pathway or vice versa, ODQ together with SQ 22536 are used to evaluate how the cross-linking between the two pathways may be influenced by KMUP-3 and KMUP-4. When ODQ was combined with SQ 22536 in endothelium-intact or endothelium-denuded aortic rings, there was an additive effect to diminish the vasorelaxations of KMUP-3 and KMUP-4 (accordingly illustrated in FIGS. 6(A)-6(D)).

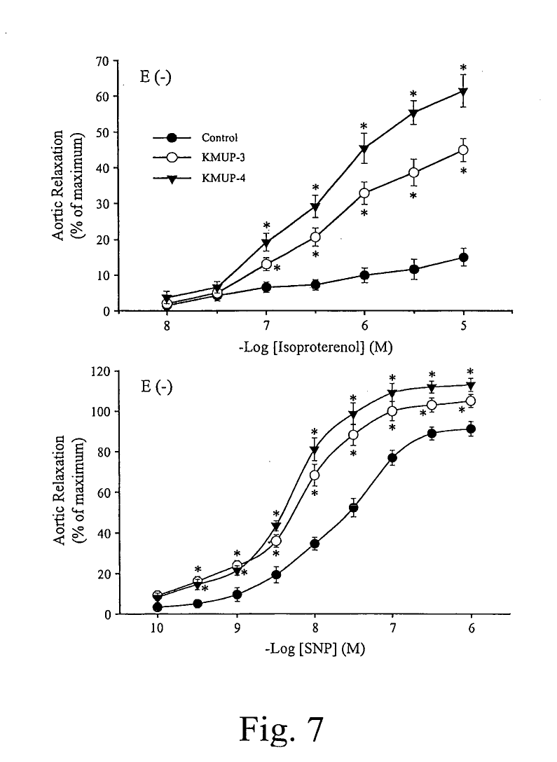

Please refer to FIG. 7. FIG. 7 shows the respective aortic relaxation diagrams induced by cumulative concentration of KMUP-3 and KMUP-4 (1 μM) on SNP- and isoproterenol-induced vasorelaxants in endothelium-denuded aortic rings precontracted by PE (10 μM) according to the present invention. Denuded aortic rings are pretreated with KMUP-3 and KMUP-4, and their responses to isoproterenol and SNP are studied, where isoproterenol is the cAMP-dependent vasodilator and SNP is the cGMP-dependent vasodilator. Both KMUP-3 and KMUP-4 (1 μM) markedly increased the potency of isoproterenol or SNP, as they shifted the concentration-response curve for isoproterenol (0.01 μM-10 mM) or SNP (1 nM-1 μM) to the left.

PDE activities were determined by the method of Hidaka and Asano (Hidaka H, Asano T., Biochim Biophys Acta., 429, 485-497, 1976). Washed human platelets were used for both PDE3 and PDE5 analyses, and human U937 cells for PDE4. Purified protein containing PDE3, PDE4, or PDE5 enzyme was resuspended in 50 mM Tris-HCl containing 5 mM MgCl₂ (pH 7.5). Subsequently, the enzyme (11.5 mg/mL, 10 mL) was incubated with Tris-HCl (80 mL), and 10 mM cGMP or cAMP substrate (final concentration 1 mM containing 0.1 μCi [³H]cGMP or [³H]cAMP) was added. After 20 minutes at 37° C., the samples were heated to 100° C. for 2 minutes. Ophiophagis Hannah snake venom (10 mg/mL, 10 mL) was then added and incubated at 37° C. for 10 minutes to convert the 5′-GMP and 5′-AMP to the uncharged nucleosides guanosine and adenosine, respectively. An ion-exchange resin (200 mL) was added to bind all unconverted cGMP or cAMP. After centrifuging, the supernatant was removed for determination of radiolabeled guanosine or adenosine by a liquid scintillation counter.

Human umbilical vein endothelial cells (HUVECs, American Type Culture Collection, Rockville, Md.) were cultured in F12 nutrient mixture medium supplemented with 10% FBS, 1.6 mM L-glutamine, 30 mg/mL endothelial cell growth supplement, 100 U/mL penicillin, 100 mg/mL streptomycin, and 10 U/mL heparin. HUVECs of passages 3-5 were used for all experiments.

The enzyme inhibitory activities of KMUP-3 (10 mM) for PDE3, PDE4, and PDE5 were 55±3.1%, 48±2.4%, and 48±2.6% (n=3), respectively. Additionally, KMUP-4 (10 mM) for PDE3, PDE4, and PDE5 activities were 56±2.8%, 33±1.7%, and 15±3.1% (n=3), respectively. Under this condition, theophylline (10 mM) was used as a reference agent, and its inhibitory actions for PDE3, PDE4, and PDE5 were 8±1.0%, 8±1.2%, and 12±2.1% (n=3), respectively.

Intracellular cGMP and cAMP levels in RASMCs were described previously (Wu B N, Lin R J, Lin C Y, et al., Br J Pharmacol., 134, 265-274, 2001). In brief, cells were grown in 24-well plates (10⁵ cells/well). At confluence, monolayer cells are washed with phosphate buffer solution (PBS) and then incubated with KMUP-3, KMUP-4, or other PDE inhibitors (10 or 100 μM) for 20 minutes. Incubation is terminated by the addition of 10% trichloroacetic acid (TCA). Cell suspensions are sonicated and then centrifuged at 2500 g for 15 minutes at 4° C. To remove TCA, the supernatants are extracted 3 times with 5 volumes of water-saturated diethyl ether. Then, the supernatants are lyophilized, and cGMP or cAMP of each sample is determined by using commercially available radioimmunoassay kits (Amersham Pharmacia Biotech, Buckinghamshire, England).

The results are expressed as mean±SEM. Statistical differences are determined by independent and paired Student t-test in unpaired and paired samples, respectively. Whenever a control group was compared with more than 1 treated group, the 1-way ANOVA or 2-way repeated-measures ANOVA was used. When the ANOVA manifested a statistical difference, the Dunnett or Tukey test is applied. A P-value of less than 0.05 is considered to be significant in all experiments. Analysis of the data and plotting of the figures are done with the aid of software (SigmaPlot Version 8.0 and SigmaStat Version 2.03, Chicago, Ill.) run on an IBM-compatible computer.

Please refer to Table 1. Table 1 is the effects of KMUP-3, KMUP-4, milrinone, rolipram and Zaprinast on cAMP and cGMP levels in RASMCs. The basal values of cGMP and cAMP are 32.14±2.61 fmol/10⁵ cells and 6.31±0.53 pmol/10⁵ cells (n=3) in RASMCs, respectively. KMUP-3, KMUP-4, and the PDE5 inhibitor zaprinast (100 μM) significantly increased the cGMP levels, but this is not observed for milrinone, the selective PDE3 inhibitor, and rolipram, the PDE4 inhibitor. Likewise, KMUP-3, KMUP-4, milrinone, and rolipram (10 μM) significantly elevated cAMP contents, but this is not found with zaprinast (10 μM). The rises of cGMP as a result of KMUP-3 and KMUP-4 were fully eliminated by pretreatment with ODQ (10 μM). Increased cAMP from KMUP-3 and KMUP-4 were partly attenuated by SQ 22536 (100 μM).

TABLE 1
Effects of KMUP-3, KMUP-4, Milrinone, Rolipram, or
Zaprinast on cAMP and cGMP Levels in RASMCs

| | | cGMP^{b} (fmol/10⁵ |
|---|---|---|
| Treatment | cAMP^{a} (pmol/10⁵ Cells) | Cells) |
| Vehicle | 6.19 ± 0.36 | 30.97 ± 2.71 |
| KMUP-3 | 16.18 ± 1.15* | 64.51 ± 4.68* |
| KMUP-4 | 15.42 ± 1.63* | 55.28 ± 5.69* |
| Milrinone | 16.24 ± 1.23* | 30.96 ± 2.96 |
| Rolipram | 18.53 ± 1.10* | 31.35 ± 2.51 |
| Zaprinast | 6.50 ± 0.45 | 50.82 ± 3.89* |

Each value represents the mean ± SEM of 3 independent experiments.
*P < 0.05 as compared with the vehicle, ANOVA followed by Dunnett test.
^{a}Measured from 10 μM of test agent each.
^{b}Measured from 100 μM of test agent each.

Please refer to Table 2. Table 2 is the effects of KMUP-3, KMUP-4, sildenafil (100 μM) on SNP (100 μM)-induced release of intracellular cGMP, respectively. As indicated in Table 3, KMUP-3, KMUP-4, and sildenafil (100 μM), the known PDE5 inhibitor, enhance the releases of intracellular cGMP by SNP (100 μM).

TABLE 2
Effects of KMUP-3 or KMUP-4 on cAMP and cGMP
Levels in RASMCs in the Absence and Presence of SQ 22536 (100 μM)
or ODQ (10 μM)

| Treatment | cAMP (pmol/10⁵ Cells)^{a} |
|---|---|
| Vehicle | 6.25 ± 0.38 |
| SQ 22536 | 6.41 ± 0.41 |
| KMUP-3 16.28 6 1.20* | 16.28 ± 1.20* |
| KMUP-3 plus SQ 22536 | 10.30 ± 0.82*† |
| KMUP-4 | 15.54 ± 1.60* |
| KMUP-4 plus SQ 22536 | 9.69 ± 0.75*† |
| | cGMP (fmol/10⁵ cells)^{b} |
| Vehicle | 30.85 ± 2.85 |
| ODQ | 29.11 ± 2.53 |
| KMUP-3 | 65.02 ± 4.75* |
| KMUP-3 plus ODQ | 34.26 ± 2.89† |
| KMUP-4 | 55.35 ± 5.60* |
| KMUP-4 plus ODQ | 32.10 ± 2.63† |

Each value represents the mean ± SEM of 3 independent experiments.
*P < 0.05 as compared with the vehicle, ANOVA followed by Dunnett test.
†P < 0.05 as compared with KMUP-3 or KMUP-4.
^{a}Measured from 10 μM of test agent each.
^{b}Measured from 100 μM of test agent each.

TABLE 3
Effects of KMUP-3, KMUP-4, or Sildenafil (100 μM)
on SNP (100 μM)-Induced Release of Intracellular cGMP

| Treatment | cGMP (fmol/10⁵ cells)^{b} |
|---|---|
| Vehicle | 30.92 ± 2.80 |
| SNP | 50.87 ± 4.35 |
| KMUP-3 | 62.50 ± 5.21* |
| Sildenafil | 64.51 ± 4.58* |
| KMUP-4 | 55.10 ± 5.59* |
| SNP plus Sildenafil | 90.49 ± 8.96*† |
| SNP plus KMUP-3 | 94.57 ± 7.85*† |
| SNP plus KMUP-4 | 81.24 ± 6.90*† |

Each value represents the mean ± SEM of 3 independent experiments.
*P < 0.05 as compared with the vehicle, ANOVA followed by Dunnett test.
†P < 0.05 as compared with sildenafil, KMUP-3, or KMUP-4.

Enhanced eNOS Expression in HUVEC

Under normal condition, the intracellular NO activates sGC, which converts GTP into cGMP to relax smooth muscle. Nitric oxide synthase has three isomers including nNOS existing in nerve cells, eNOS existing in endothelia cells, and iNOS existing in macrophages. The nNOS and eNOS produce a small amount, and are Ca²⁺-dependent and consititutive-expressed. Therefore, the eNOS expression in HUVEC is enhanced which could be supported for the effect induced by KMUP-3 and KMUP-4.

Endothelial cells were processed, and eNOS protein abundance was determined by Western blot analysis using anti-eNOS antibody (BD Transduction Laboratories, San Diego, Calif.) in a manner that was similar to that described in our previous study.

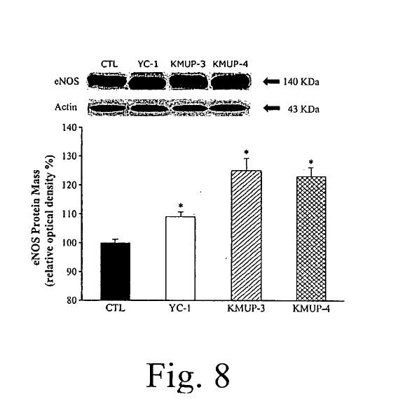

Please refer to FIG. 8. FIG. 8 is a densitometry result of the expression of eNOS in HUVECs induced by YC-1, KMUP-3, and KMUP-4 (0.1 μM) according to the present invention. Immunoreactivity of eNOS protein is detected in preparation for HUVEC. A time-dependent increase in eNOS expression was observed with 0.1 μM KMUP-3 and KMUP-4, and the maximal protein expression was achieved at 18 hours thereafter in a trend of decrease (unpublished data). The expression of eNOS protein was significantly increased by treating with 0.1 mM KMUP-3, KMUP-4, and a sGC activator YC-1 for 18 hours.

### EXAMPLE 1

The Synthesis of KMUP-3

Dissolve 1 mol theophylline into 2 mol 1,2-dibromoethane buffer to form a mixture. Stir the mixture until the temperature is raised to 100° C. on the mantle heater. After theophylline completely dissolves in the buffer, 125 ml, 1.6 N NaOH is added thereinto to react for 5-8 hours at approximately 100° C. Next, the mentioned reaction solution is filterated out the precipitated white NaBr under a reduced pressure and then concentrated to obtain an oil-like solution. Purify the oil-like solution with a solvent mixture of n-hexane and ethylacetate by a column having a packing gel of silica gel 60 to obtain an oil-like compound A. Dissolve the compound A in methanol and add piperazine for performing a reflux reaction to obtain a reaction solution. Further, the mentioned reaction solution is under a reduced pressure and then concentrated to obtain a first coarse crystal. Recrystallize the first coarse crystal with methanol and purify the first coarse crystal by a column having a packing gel of silica gel 60 to obtain a crystal compound B. Dissolve the crystal compound B in methanol to form a first solution. Then, dissolve 4-chloronitrobenzene in the first solution to form a second solution and perform a reflux reaction to obtain KMUP-3. KMUP-3 is further recrystalized in methanol.

The physical properties of KMUP-3:

¹H NMR (CDCl₃): δ 3.39 (s, 3H, NCH₃), 3.57 (s, 3H, NCH₃), 2.82 (t, 2H, NCH₂), 4.43 (t, 2H, NCH₂), 2.64 (t, 4H, 2×CH₂), 3.10 (t, 4H, 2×CH₂), 6.77-6.81 (m, 2H, 2×Ar—H), 7.15-7.19 (m, 2H, 2×Ar—H), 7.65 (s, 1H, imidazole-H); IR (KBr): 748.54 (ArC—Cl) & 1685.26 (C═O) cm⁻¹; MS (m/s): 403 (Scan FAB⁺). Anal. (C₁₉H₂₃N₄O₇)C, H, N.

### EXAMPLE 2

The Synthesis of KMUP-4

Dissolve 1 mol theophylline into 2 mol 1,2-dibromoethane buffer to form a mixture. Stir the mixture until the temperature is raised to 100° C. on the mantle heater. After the theophylline completely dissolves in the buffer, 125 ml, 1.6 N NaOH is added thereinto to react for 5-8 hours at approximately 100° C. Next, the mentioned reaction solution is filterated out the precipitated white NaBr under a reduced pressure and then concentrated to obtain an oil-like solution. Purify the oil-like solution by a column having a packing gel of silica gel 60 to obtain a compound A. Dissolve the compound A in methanol and add piperazine for performing a refluxation to obtain a reaction solution. Further, the mentioned reaction solution is under a reduced pressure and then concentrated to obtain a first coarse crystal. Recrystallize the first coarse crystal with methanol and purify the second coarse crystal by a column having a packing gel of silica gel 60 to obtain a crystal compound B. Dissolve the crystal compound B in methanol to form a first solution. Then, dissolve 2-chloronitrobenzene in the first solution to form a second solution and perform a reflux reaction to obtain KMUP-4. KMUP-4 is further recrystalized in methanol.

The physical properties of KMUP-4:

¹H NMR (CDCl₃): δ 3.60 (s, 3H, NCH₃), 3.42 (s, 3H, NCH₃), 4.45 (t, 2H, NCH₂), 2.85 (t, 2H, NCH₂), 2.70 (t, 4H, 2×CH₂), 3.40 (t, 4H, 2×CH₂), 6.82 (m, 2H, 2×Ar—H), 8.10 (m, 2H, 2×Ar—H), 7.69 (s, 1H, imidazole-H); IR (KBr): 1323.58 (NO₂) & 1657.48 (C═O) cm⁻¹; MS (m/s): 414 (Scan FAB⁺). Anal. (C₁₉H₂₃N₄O₇)C, H, N.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A substance for enhancing an aortic smooth muscle relaxation, being one selected from the group consisting of a compound of formula (I), a pharmaceutical acceptable therefrom and a solvate therefrom, 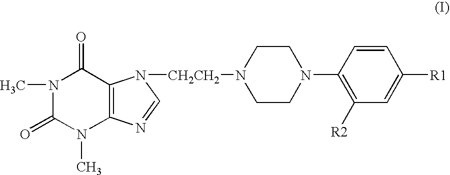
wherein either of R1 and R2 is one of a hydrogen and a nitro group.

2. The substance as claimed in claim 1, wherein R1 is a nitro group.

3. The substance as claimed in claim 1, wherein R2 is a nitro group.

4. A pharmaceutical composition for enhancing an aortic smooth muscle relaxation, comprising a substance of claim 1 and one selected from the group consisting of a pharmaceutical excipient, a diluent and a carrier.

5. A method for synthesizing a compound for enhancing an aortic smooth muscle relaxation, comprising the following steps:
(1) dissolving theophylline into 1,2-dibromoethane to form a mixture;
(2) adding NaOH into the mixture to obtain an oil-like solution;
(3) purifying the oil-like solution to obtain an oil-like compound;
(4) adding piperazine to react with the oil-like compound to obtain a first coarse crystal;
(5) recrystallizing and purifying the first coarse crystal to obtain the first crystal compound;
(6) dissolving the first crystal compound in a solvent to form a first solution;
(7) dissolving 2-chloronitrobenzene and 4-chloronitrobenzene respectively in the first solution to form a second solution;
(8) obtaining the compound from the second solution.

6. The method as claimed in claim 5, wherein the reaction temperature of the step (2) is performed at approximately 90-120° C.

7. The method as claimed in claim 5, wherein the reaction temperature of the step (2) is performed at 100° C.

8. The method as claimed in claim 5, wherein a first methanol solution is used for the step (3) and a second methanol solution is used for the step (5).

9. The method as claimed in claim 5, wherein a solvent mixture of n-hexane and ethylacetate is used for the step (3).

10. The method as claimed in claim 5, wherein the steps (3) and (5) are performed by a column.

11. The method as claimed in claim 5, wherein the column has a packing gel being silica gel 60.

12. The method as claimed in claim 5, wherein the solvent in the step (6) is methanol.

13. The method as claimed in claim 5, wherein the step (8) is performed by a refluxing.

14. A method for enhancing an aortic smooth muscle relaxation of a mammal, comprising:
administrating to the mammal one of the group consisting of a compound of formula (I), a salt thereof and a solvate thereof, and a pharmaceutical carrier, 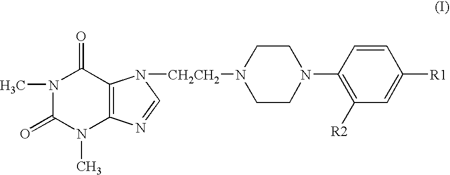
wherein either of R1 and R2 is one of a hydrogen and a nitro group.
